(19) United States
(12) Patent Application Publication
(10) Pub. No.: US 2015/0265318 A1
(43) Pub. Date: Sep. 24, 2015

(54) SYSTEM FOR ENHANCING BONE GROWTH ON ORTHOPEDIC IMPLANTS

(71) Applicant: HAMID ABBASI, Alexandria, MN (US)

(72) Inventor: HAMID ABBASI, Alexandria, MN (US)

(21) Appl. No.: 14/220,487

(22) Filed: Mar. 20, 2014

(51) Int. Cl. A61B 17/70

(52) U.S. Cl. CPC A61B 17/7061; A61B 17/7032

(57) ABSTRACT

A system for implantation in a patient's body may comprise at least one screw configured for threaded insertion into a bone of the patient and having an exterior surface, and at least one rod configured for attaching to a said screw and having an exterior surface. Hollow spaces, passages, interstices or the like may be formed in at least one of the screw and rod and are in communication with the exterior surface of the structure and extend into the structure. A substance is positioned in at least some of the hollow spaces, passages or interstices, and the substance may have a characteristic of promoting bone growth.

### BACKGROUND

### Field

The present disclosure relates to orthopedic implants and more particularly pertains to a new system for enhancing bone growth on orthopedic implants to help promote bonding of the implanted structures to the bone to which the structures are attached.

### SUMMARY

In one aspect, the disclosure relates to a kit for implantation in the body of a patient to stabilize the spine of the patient, and the kit may comprise at least one screw configured for threaded insertion into a bone of the patient. The screw has an exterior surface, a head portion and a body portion with the body portion having threads formed thereon. The kit may also comprise at least one rod configured for attaching to a said screw, and the rod has an exterior surface. A plurality of hollow spaces may be formed in at least one structure of the group of structures comprising the at least one screw and the at least one rod, and the hollow spaces may be in communication with the exterior surface of the structure and extend into the structure. A substance is positioned in at least some of the plurality of hollow spaces, and the substance has a characteristic of promoting bone growth.

In another aspect, the disclosure relates to a kit for implantation in the body of a patient to stabilize the spine of the patient, and the kit may comprise at least one screw configured for threaded insertion into a bone of the patient. The screw has an exterior surface, a head portion and a body portion with the body portion having threads formed thereon. The kit may also comprise at least one rod configured for attaching to a said screw, and the rod has an exterior surface. At least one passage may be formed in at least one structure of the group of structures comprising the at least one screw and the at least one rod, and the passage may be in communication with the exterior surface of the structure and extend into the structure. A substance is positioned in the passage of the structure, and the substance has a characteristic of promoting bone growth.

In still another aspect, the disclosure relates to a spinal stabilizing assembly for implantation in the body of a patient to stabilize the spine of the patient. The assembly may comprise at least a pair of screws configured for threaded insertion into a bone of the patient. The screws have an exterior surface, and may include a head portion and a body portion with the body portion having threads formed thereon. The assembly may also comprise at least one rod mountable on the pair of screws to join them together, and the rod also has an exterior surface. A plurality of hollow spaces may be formed in at least one structure of the group of structures comprising the screws and the at least one rod, and the hollow spaces being in communication with the exterior surface of the structure and extend into the structure. A substance is positioned in at least some of the plurality of cavities, and the substance having a characteristic of promoting bone growth.

There has thus been outlined, rather broadly, some of the more important elements of the disclosure in order that the detailed description thereof that follows may be better understood, and in order that the present contribution to the art may be better appreciated. There are additional elements of the disclosure that will be described hereinafter and which will form the subject matter of the claims appended hereto.

In this respect, before explaining at least one embodiment or implementation in greater detail, it is to be understood that the scope of the disclosure is not limited in its application to the details of construction and to the arrangements of the components, and the particulars of the steps, set forth in the following description or illustrated in the drawings. The disclosure is capable of other embodiments and implementations and is thus capable of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present disclosure. It is important, therefore, that the claims be regarded as including such equivalent constructions insofar as they do not depart from the spirit and scope of the present disclosure.

The advantages of the various embodiments of the present disclosure, along with the various features of novelty that characterize the disclosure, are disclosed in the following descriptive matter and accompanying drawings.

### BRIEF DESCRIPTION OF THE DRAWINGS

The disclosure will be better understood and when consideration is given to the drawings and the detailed description which follows. Such description makes reference to the annexed drawings wherein:

FIG. 1 is a schematic side view of a screw structure of the new system for enhancing bone growth on orthopedic implants according to the present disclosure.

FIG. 2 is a schematic side view of an enlarged portion of the screw structure, according to an illustrative embodiment.

FIG. 3 is a schematic fragmentary sectional view of the screw structure showing a hollowed out space with substance situated therein, according to an illustrative embodiment.

FIG. 4 is a schematic side view of a screw structure and rod structure of the system, illustrating channels and passages of an illustrative embodiment.

FIG. 5 is a schematic side sectional view of the screw and rod structures in an assembled condition with the channels and passages having the substance positioned therein, according to an illustrative embodiment.

FIG. 6 is a schematic end view of a rod structure having interstices formed therein for receiving a substance, according to an illustrative embodiment.

FIG. 7 is a schematic side view of a portion of a screw structure and a portion of a rod structure having interstices formed thereon for receiving a substance, according to an illustrative embodiment.

FIG. 8 is a schematic sectional view of an enlarged portion of the surface of one of the structures of the disclosure showing the interstices formed therein.

FIG. 9 is a schematic sectional view of a portion of the surface of one of the structures having the interstice formed therein and substances applied thereto.

FIG. 10 is a schematic end view of a rod structure having a channel with an optional configuration.

### DETAILED DESCRIPTION

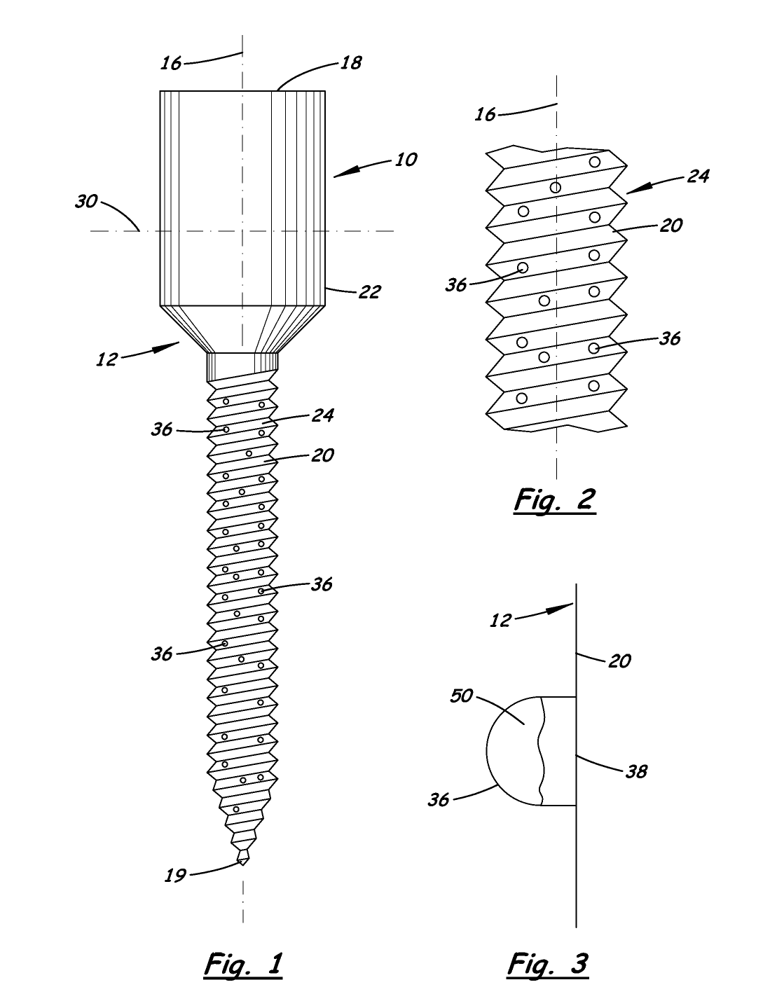

With reference now to the drawings, and in particular to FIGS. 1 through 10 thereof, a new system for enhancing bone growth on orthopedic implants embodying the principles and concepts of the disclosed subject matter will be described.

In general, the disclosure relates to structures for securing to bones in a mammalian body, and in particular may be implemented on a human patient although the application of the disclosure is not so limited. Aspects of the disclosure may be utilized to connect two of more bones of the patient together, such as, for example, in immobilizing two vertebrae of the patient's spine with respect to each other.

In greater detail, illustrative embodiments of the structures will be described. Some illustrative embodiments may be utilized as a spine stabilization structure 10 for implantation in a patient and connection to two or more vertebrae of the patient's spine. The spine stabilization structure 10 may comprise one or more screws 12, and one or more rods 14 that may be connected together in various configurations that will be evident from the following description.

The screw 12 may be configured for threaded insertion into a bone of the patient, such as a preformed hole in the bone, and in some implementations may be configured for mounting on the pedicle of a vertebra of the patient (see, e.g., FIG. 1). Thus, in general the screw may an overall shape that is similar to known pedicle screws, although this is not critical and other forms may be utilized. The screw 12 is typically elongated with a longitudinal axis 16 and has a first longitudinal end 18 and a second longitudinal end 19. The screw has an exterior surface 20, and the rod also has an exterior surface 21.

The screw 12 may include a head portion 22 which is located toward the first longitudinal end 18 of the screw, and may also include a body portion 24 located toward the second longitudinal end 19 of the screw. In some embodiments, the head portion 22 and the body portion 24 of the screw may be able to be removed from each other and then reconnected. The head 22 and body 24 portions may be movable with respect to each other when connected. The head portion 22 may define a cavity 24, and a channel 26 for receiving a portion of a rod 14 therethrough (see, e.g., FIG. 5). The channel 26 may extend through the cavity along a transverse axis 30 extending substantially transverse to the longitudinal axis 16 of the screw. In some embodiments, the cavity 24 may be defined by inner surfaces 32 which may have threads formed thereon. A rod securing element 34 may be positionable in the cavity, and may have an outer peripheral surface with threads formed thereon for engaging the threads on the inner surfaces of the head portion. A portion of the rod 14 may be positioned through the channel 26 and in the cavity 24 and the rod securing element 34 may be threaded into the cavity to secure the rod to the head portion. Additional suitable aspects of the design are described in my copending patent application Ser. No. 14/043,510 filed Oct. 1, 2013 which is hereby incorporated by reference in its entirety.

The body portion 24 of the screw may be configured to insert or screw into a hole formed in the vertebra, and may have a portion of the exterior surface 20 on the body portion being threaded for that purpose (see, e.g., FIGS. 1, 2, 4 and 5). The body portion may terminate in a tip at the second longitudinal end 19 of the screw.

In some embodiments of the disclosure, a significant aspect relates to characteristics of portions of the exterior surfaces 20, 21 of the screws and rods, although these characteristics are not necessarily present in both the screws and rods. Those portions of the exterior surfaces of the may include one or more openings 38 in the surface, and in some embodiments the openings 38 open into hollowed out spaces 36 in the interior of the that may be capable of receiving and holding a quantity of substance described elsewhere in this disclosure. (see e.g., FIGS. 1 through 3). The spaces 36 may occupy areas on the exterior surface that correspond to the openings 38. The spaces 36 may be generally closed to the interior or have a blind end opposite of the opening 38. The openings 38 may be round or regular in shape, but could also be irregular in shape. The spaces may form cavities or recesses or indentations or pits in the surface, and may be evenly distributed across the surface of the screw or rod or may be irregularly distributed on the surface. The hollowed out spaces 36 may be positioned at functionally-important or significant locations on the surface for the purposes described herein. Further, the sizes and shapes of the areas on the exterior surface may vary, and may be relatively larger or smaller to suit a particular application. For example, in implementations in which a substance is positioned in the space, the size may be relatively larger than implementations in which the space that lacks substance positioned therein.

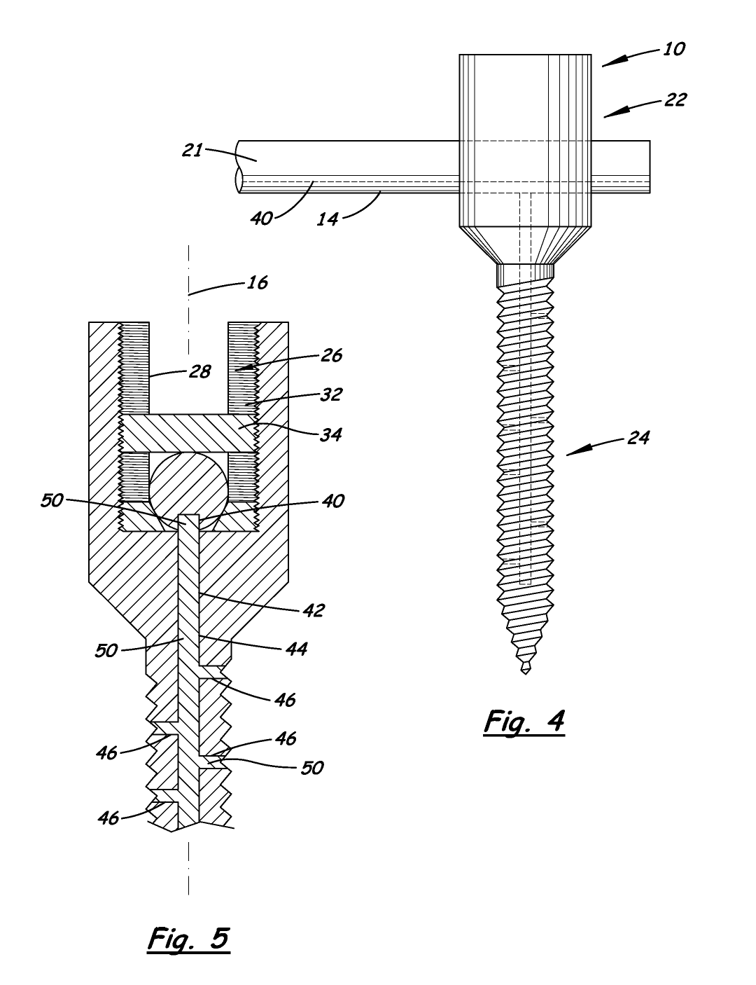

While the hollowed-out spaces 36 may be implemented on the exterior surfaces 20, 21 of the screws and rods, in some implementations a groove or channel 40 may be formed on the exterior surface of the rod which is capable of receiving and holding a quantity of a substance (see e.g., FIGS. 4, 5 and 10). The channel 40 may extend in a longitudinal direction of the rod, but other orientations may be employed, and may extend for the entire length of the rod or only portions of the rod's length. The spaces and channels may be formed by any suitable means, such as removal of material from the formed screw or rod, or even forming the screw or rod with the spaces therein. The spaces may be formed in both the head and body portions of the screw, but in some of the most preferred embodiments the spaces may be primarily formed in the body portion, and may be located among the threads formed on the exterior surface of the body portion. The shape of the groove or channel 40 may vary, and illustratively may have a rectangular shape (see, e.g., FIG. 5) or a triangular shape (see, e.g., FIG. 10). In some embodiments, the depth of the channel may be up to approximately one quarter of the width or diameter of the rod structure, although other depths may be used and the depth may be greater than the one-quarter diameter depth. For example, the depth of the channel may be up to one half of the diameter of the rod structure to create a channel with greater capacity, although the strength and rigidity of the rod structure should not be compromised.

In some embodiments of the disclosure, a significant aspect relates to one or more passages 42 that may be formed in the screw 12 and that are capable of receiving and holding a quantity of substance described elsewhere in this disclosure (see e.g., FIGS. 4 and 5). The passage 42 may pass entirely through the screw so that the passage terminates in two or more openings in the exterior surface, or in some cases may have a blind end with a single opening. Illustratively, the passage 42 may have a central portion 44 that extends along at least a portion of the longitudinal axis 16 of the screw, and in some embodiments, the passage 42 also includes at least one radiating portion 46 that radiates outwardly from the central portion 44. The central portion 44 and the radiating portions 46 may be in communication with each other. While passages may be located in either of the head or body portions of the screw, many highly preferable embodiments have the passages primarily located in the body portion of the screw with limited numbers of passages in the head portion.

In some of the most preferred embodiments of the disclosure, the hollows or passages of the screws and/or rods may be at least partially filled with or occupied by a substance 50 that has desirable characteristics for promoting the function and performance of the screws and rods in the body, or at least the overall purpose of the implantation of the structure in the body. For example, the substance may promote the bonding of the structures to the bone to which the structure are mechanically connected, or otherwise associated, and in particular forming a bond formed by biological elements. The substances may also promote the growth of bone about the structures (e.g., screws and rods) when implanted in the body. The substances may be applied to the structures before the implantation of the structures in the body of the patient, and may be applied well before the time of implantation or just before the time of implantation, or even after implantation. For example, in embodiments of the structure that have channels or grooves formed therein, the substance may be injected or otherwise filled into the channels and grooves after the screw and/or rod structures have been implanted into the body which may facilitate the use of forms of the substance (e.g., liquids or gels) that may be difficult or impossible to apply prior to implantation. In some highly preferred embodiments, the substance comprises a biologic substance.

Some suitable substances have the characteristic of attracting osteoblast cells in the body of the patient to the location or site of the substance on the screw or rod structure that help form bone in and about the screw and rod structures. For example, biologics may perform this functionality, and one example of a suitable biologic is demineralized bone matrix. Other substances with similar attractive capabilities include tri-calcium phosphate, although the disclosure is not limited to any specific compound listed here. There are other substances that are capable of enhancing the growth of bone in and around the areas of the screw and rod structures that hold the substance in the respective structure.

Screw and rod structures that include the substances situated in the various hollows and passages thus encourage and enhance the growth of bone in and around the screws and rods in a beneficial manner that function to enhance the bond between the structures and the patient's bone, and well as strengthening of the overall structure implanted into the body of the patient. It should be recognized that all or only some of the spaces may hold a substance, and in some implementations none of the spaces may hold the substance. It should be recognized that the substance may be a flowable mass such as a putty or gel or liquid that may be injected or otherwise forced into the passages or channels or grooves prior to, just prior to, or after implantation of the structure in the body, thus providing an in situ implementation. For example, referring to FIG. 5, prior to the installation of the rod structure and the rod securing element, an injection device may be used to inject the substance into the passage through the upper end or port of the passage so that the substance may move into the branches of the passage toward the openings at the end of the branches. In some embodiments, the injection device may comprise a tool used to rotate the screw, such as a screwdriver, or a syringe or other device able to forcibly move the substance into the passage.

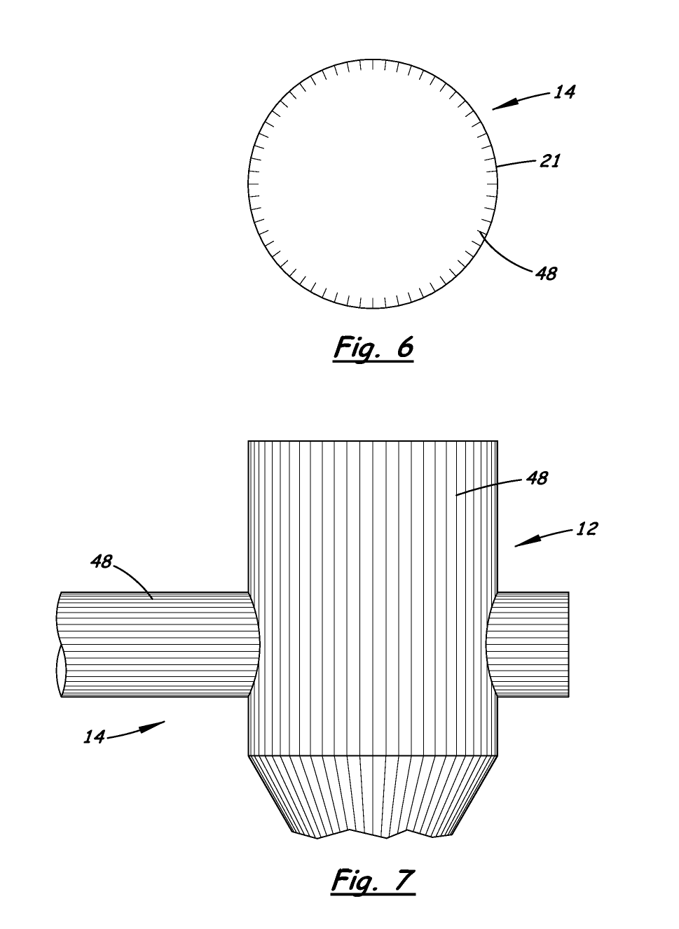

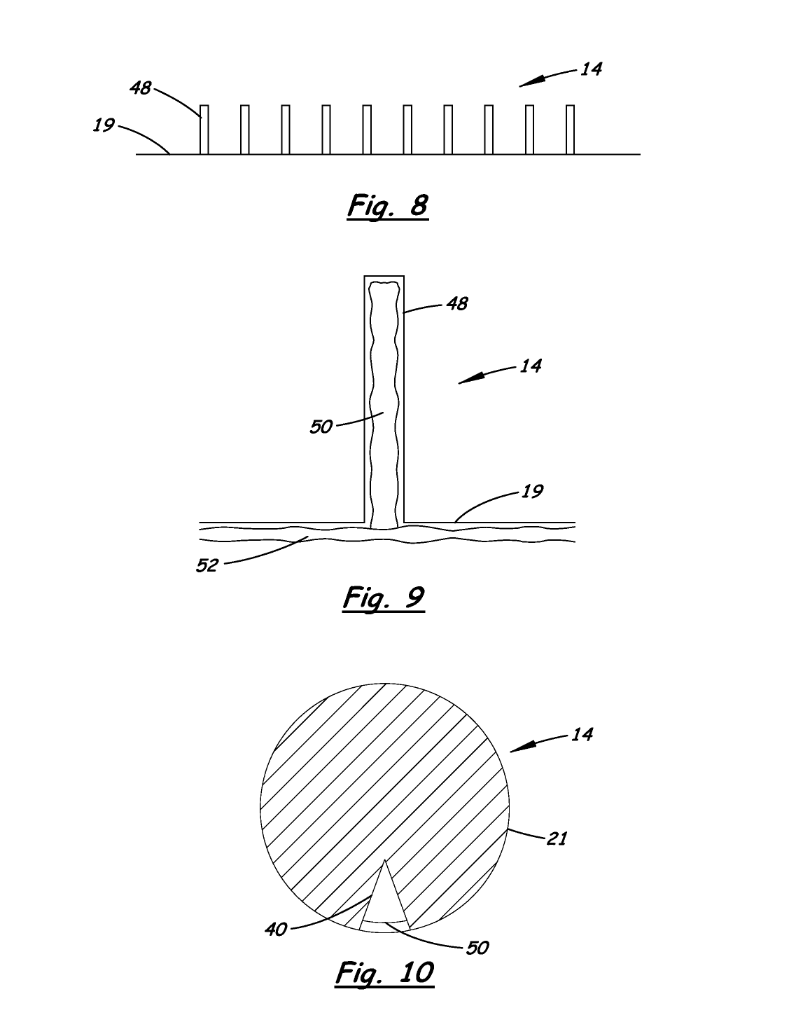

In still other embodiments, the exterior surface of the screw and/or rod may be roughened or etched or furrowed to create interstices 48 formed on the surface that increase the surface area of the exterior surface and create areas which better hold a substance on the surface (see e.g., FIGS. 6 through 9). The interstices may be in the form of small grooves that are cut into the surface by cutting away material from the surface using any suitable technology such as, for example, a laser, a mill, a water jet or other applicable technology. In some embodiments, the interstices are relatively thin and narrow in comparison to the depth of the interstices. Although the interstices are illustrated as being formed on the surface 19 of the rod structure 14, the interstices are also useful on the screw structure 12. The interstices may be formed, for example, on portions of the exterior surface of the rod structure (see, e.g., FIG. 6) and/or the screw structure (see, e.g., FIG. 7) and may extend linearly, although this is not critical to the disclosure. In some embodiments, the interstices may have the form of shallow slots or grooves (see, e.g., FIGS. 8 and 9) that are suitable to receive a substance. The grooves may have various sizes and shapes suitable for the particular application, and may form smaller microgrooves or larger macro-grooves that have different capacities for holding the substance.

A substance 50 or substances may be applied to the exterior surface on which the interstices have been created in order to foster bone material growth on, or bonding with, the surface. In some highly preferred implementations, the surface with the interstices may have a collagen substance applied to the surface and the collagen may form a coating layer on the surface including the surface inside the interstices. Subsequent to coating the surface with collagen, and preferably just prior to implantation of elements of the structure, a further second substance 52 may be added to the surface of the structure to enhance bone growth or bonding. The further substance may include bone morphogenetic protein (BMP), although other substances may be employed, to impregnate the interstices.

It should be appreciated that in the foregoing description and appended claims, that the terms “substantially” and “approximately,” when used to modify another term, mean “for the most part” or “being largely but not wholly or completely that which is specified” by the modified term.

It should also be appreciated from the foregoing description that, except when mutually exclusive, the features of the various embodiments described herein may be combined with features of other embodiments as desired while remaining within the intended scope of the disclosure.

Further, those skilled in the art will appreciate that the steps shown in the drawing figures may be altered in a variety of ways. For example, the order of the steps may be rearranged, substeps may be performed in parallel, shown steps may be omitted, or other steps may be included, etc.

With respect to the above description then, it is to be realized that the optimum dimensional relationships for the parts of the disclosed embodiments and implementations, to include variations in size, materials, shape, form, function and manner of operation, assembly and use, are deemed readily apparent and obvious to one skilled in the art in light of the foregoing disclosure, and all equivalent relationships to those illustrated in the drawings and described in the specification are intended to be encompassed by the present disclosure.

Therefore, the foregoing is considered as illustrative only of the principles of the disclosure. Further, since numerous modifications and changes will readily occur to those skilled in the art, it is not desired to limit the disclosed subject matter to the exact construction and operation shown and described, and accordingly, all suitable modifications and equivalents may be resorted to that fall within the scope of the claims.

I claim:

1. A kit for implantation in the body of a patient to stabilize the spine of the patient, the kit comprising:
at least one screw configured for threaded insertion into a bone of the patient, the screw having an exterior surface, the screw including a head portion and a body portion, the body portion having threads formed thereon;
at least one rod configured for attaching to a said screw, the rod having an exterior surface;
a plurality of hollow spaces formed in at least one structure of the group of structures comprising the at least one screw and the at least one rod, the hollow spaces being in communication with the exterior surface of the structure and extending into the structure; and
a substance positioned in at least some of the plurality of hollow spaces, the substance having a characteristic of promoting bone growth.

2. The kit of claim 1 wherein the substance comprises a biologic substance.

3. The kit of claim 2 wherein the biologic substance comprises a demineralized bone matrix.

4. The kit of claim 2 wherein the biologic substance attracts osteoblast cells when implanted in the body.

5. The kit of claim 1 wherein the hollow spaces are located on portions of the exterior surface that contact bone when the structure is implanted in the body of a patient.

6. A kit for implantation in the body of a patient to stabilize the spine of the patient, the kit comprising:
at least one screw configured for threaded insertion into a bone of the patient, the screw having an exterior surface, the screw including a head portion and a body portion, the body portion having threads formed thereon;
at least one rod configured for attaching to a said screw, the rod having an exterior surface;
at least one passage formed in at least one structure of the group of structures comprising the at least one screw and the at least one rod, the passage being in communication with the exterior surface of the structure and extending into the structure; and
a substance positioned in the passage of the structure, the substance having a characteristic of promoting bone growth.

7. The kit of claim 6 wherein the substance comprises a biologic substance.

8. The kit of claim 7 wherein the biologic substance comprises a demineralized bone matrix.

9. The kit of claim 7 wherein the biologic substance attracts osteoblast cells when implanted in the body.

10. The kit of claim 6 wherein the passage opens to portions of the exterior surface that contact bone when the structure is implanted in the body of a patient.

11. A spinal stabilizing assembly for implantation in the body of a patient to stabilize the spine of the patient, the assembly comprising:
at least a pair of screws configured for threaded insertion into a bone of the patient, the screws having an exterior surface, the screw including a head portion and a body portion, the body portion having threads formed thereon;
at least one rod mountable on the pair of screws to join them together, the rod having an exterior surface;
a plurality of hollow spaces formed in at least one structure of the group of structures comprising the screws and the at least one rod, the hollow spaces being in communication with the exterior surface of the structure and extending into the structure; and
a substance positioned in at least some of the plurality of cavities, the substance having a characteristic of promoting bone growth.

12. The assembly of claim 11 wherein the substance comprises a biologic substance.

13. The assembly of claim 12 wherein the biologic substance comprises a demineralized bone matrix.

14. The assembly of claim 12 wherein the biologic substance attracts osteoblast cells when implanted in the body.

15. The assembly of claim 11 wherein the hollow spaces are located on portions of the exterior surface that contact bone when the structure is implanted in the body of a patient.

16. The assembly of claim 11 wherein the hollow spaces comprise interstices formed in the exterior surface.

17. The assembly of claim 16 wherein the interstices are thin compared to a depth of the interstices.
